(19) United States
(12) Patent Application Publication
(10) Pub. No.: US 2015/0157350 A1
(43) Pub. Date: Jun. 11, 2015

(54) Nerve decompression scissors

(71) Applicant: Michael Graham, Shelby Township, MI (US)

(72) Inventor: Michael Graham, Shelby Township, MI (US)

(73) Assignee: Graham Medical Technologies LLC, Macomb, MI (US)

(21) Appl. No.: 14/078,523

(22) Filed: Nov. 12, 2013

Related U.S. Application Data
(60) Provisional application No. 61/724,906, filed on Nov. 9, 2012.

(51) Int. Cl. A61B 17/3201

(52) U.S. Cl. CPC A61B 17/3201; A61B 2017/320044

(57) ABSTRACT

A surgical instrument and method for freeing tissue in a nerve decompression operation. Tissue is freed from the area surrounding a nerve with use of the blunt tip on one jaw of the instrument. The freed tissue is then cut with the bladed region of the instrument and removed from the area surrounding the nerve while leaving the nerve untouched. The instrument includes a pair of pivotally connected arms. Each of the arms is elongated and has a free end and a handle end, the free end of each having a blade, the handle end of each having a finger loop for the surgeon to grasp. One of the free ends is longer than the other, with a blunt, rounded tip and edge for freeing tissue without cutting. The scissors are curved, to the right or left, to further insure against the nerve being nicked during the operation. The pivot between the two arms separates the two sections and allows the arms to move with respect to each other within a plane of pivotal movement so that the tissue is selectively cut along the extent of the overlap of the arms.

This application is related to U.S. application Ser. No. 12/574,699 filed Oct. 6, 2009, the contents of which are incorporated by reference, and claims benefit of U.S. Provisional Application No. 61/724,906, filed Nov. 9, 2012.

### BACKGROUND OF THE INVENTION

This invention generally relates to surgical instruments, and more specifically to a surgical instrument designed to selectively cut fibers during a nerve decompression operation. The instrument is particularly suited to decompress the posterior tibial nerve on the inner ankle.

A common condition experienced by patients is a numbness or pain, for example, in the ankle region. Alternatively, the patient may experience muscle weakness in this area or trouble maintaining balance. One common cause underlying these symptoms is nerve damage due to compression of the nerve. Typically, the nerve is compressed by tissue surrounding the nerve which has become inflamed and swollen. Whatever the cause, the pressure on the nerve must be relived or permanent nerve damage could result. In the ankle region, one common concern is “Tarsal Tunnel Syndrome.” This is caused by compression of the posterior tibial nerve by the lacinate ligament.

Nerve decompression surgery is one treatment for nerve compression. In the case of posterior tibial nerve, the pressure arising from the lacinate ligament, which passes above the nerve and presses down upon it, is relived by cutting the portion of lacinate ligament in the specific site in which above ligament passed over the nerve. One the ligament is severed it retracts from the site of the cut and the pressure on the underlying nerve is relived.

One critical aspect of nerve decompression surgery is that the surrounding tissue must be removed from the area around the nerve fibers without damaging any nerve fibers. This is particularly difficult in the case of the cutting the lacinate ligament because it is a blind operation. The ligament passing above the nerve obscures the nerve from view by the surgeon.

Different surgical techniques are employed to separate and cut tissue from around nerve fibers. Some surgical techniques rely heavily on a surgeon's skillful use of a scalpel to precisely cut away only surrounding tissue without also cutting into any nervous tissue. This has the disadvantage of requiring a very steady hand and being painstakingly slow. Moreover, there is the ever present danger of an inadvertently nick of the nerve fibers if either the scalpel or the target tissues are jostled even a small amount.

Alternatively, a surgeon can use a blunt instrument, such as a surgical probe or a forceps to separate the nerve fibers from the surrounding tissue. While this has the advantage that the blunt edge is unlikely to nick nervous tissue, there is a disadvantage. The use of two surgical instruments requires a larger incision. This in turn inevitably leads to a more damage to surrounding tissues and thus longer recovery times and more frequent post operative complications. Also, a two handed surgical procedure is more difficult. The surgeon must simultaneously coordinate the movement of both hands.

In the case of the cutting of the lacinate ligament, there is the additional problem of wound dehiscence, the opening of a wound (or the failure of a wound to properly heal) due to skin tension which pulls the two halves of the wound apart. Skin tension is particularly high in the ankle region. Accordingly, there is a long felt need for surgeons to make the smallest incision possible for cutting the lacinate ligament so as to keep the risk of wound dehiscence as small as possible.

Microsurgical techniques can be employed. While these methods result in extremely precise work, they require elaborate instrumentation and can take an extremely long time. The handles of these instruments must be connected to their functional ends through a complex gear system such that a macroscopic motion of the handle is translated to a microscopic motion of the functional end. Also a microscopic viewing system must be employed. This elaborate instrumentation makes the procedure difficult to perform, if not impossible, in a clinical or “out patient” setting. A state-of-the-art operating room is required.

A variety of specially shaped scissors have been developed for various purposes. One such scissors combines tissue dissection and surgical implantation. These scissors are designed to grasp and release a tissue, in one conformation, and dissect tissue in another conformation. The instrument is designed to replace methods of using both a scissors to open a pocket in layers of tissue and a tweezers, hemostats, or graspers to position and insert mesh or other material into the tissue pocket. The scissors also measures distances or lengths of dissected tissue and anatomical structure, eliminating the extra step of using a ruler. Each blade of the scissors has a flattened tip for blunt dissection and a non-cutting clamping portion for selectively grasping a surgical implant device. The blades pivot relatively freely between the dissection and clamping positions.

Another specially shaped instrument is designed for use with the optic nerve. This instrument clamps the optic nerve during the removal of a diseased eyeball. Such clamping of the nerve was formerly done by feel with a hemostat, which resulted in uncertainty as to the extent of tissue secured in the clamp. The optic nerve clamp has a specific size and curvature designed to reduce the loss of tissue behind the eye by facilitating isolation and clamping of the optic nerve alone and can also reduce the incidence of unintentional excisions in the back part of the eyeball and other damage to the surrounding tissue. The clamp includes a pair of jaws which are curved, with one tip shorter than the other. The end of the longer tip is tapered. The varied length in the tip portions is adapted to allow the optic nerve to be located between the ends of the shorter and longer tips. The longer tip has a smooth flat surface adjacent to the tip. The variation in tip lengths allows the surgeon to slide the instrument as needed to precisely locate the nerve before clamping it. It is also suggested that two clamps with opposite curvatures could be created, one for the left eye and one for the right. This instrument differs from the disclosed invention. For example, this instrument does not include a scissors function.

Another instrument is designed to cut one layer of tissue while leaving an underlying tissue layer unscathed. This design relies primarily on a sharp right angle turn in the tip of the scissors which allows the surgeon to cut the target tissue at an oblique angle.

None of these instruments are able to perform a nerve compression operation. They lack the ability to both separate the surrounding tissue from the nerve and dissect away the surrounding tissue.

### SUMMARY OF THE INVENTION

Disclosed herein is a surgical instrument that can perform multiple surgical tasks and requires the use of only one hand. The decompression scissors can (1) free a target tissue to be removed from nerve fiber and (2) cut away and remove the target tissue without damaging the nerve tissue.

The decompression scissors includes a pair of arms connected at a pivot point. The scissors have two arms each of which have a jaw on one side of a pivot and a handle end on the opposite side of the pivot. The portion of the longer jaw extending beyond the shorter jaw has a blunt, rounded tip and no blade along the inside edge. The portion of the longer jaws that overlaps with the shorter jaw is bladed.

In an additional embodiment of the present invention, the jaws of both arms can be curve upwards, within the plane containing the arms and handles of the scissors.

### BRIEF DESCRIPTION OF THE DRAWINGS

FIGS. 1A and 1B illustrate front and side views of a curved nerve decompression scissors.

FIGS. 2A and 2B illustrate front and side view of a nerve decompression scissors without a curve.

FIG. 3 illustrates the scissors in the context of tissue to be severed and an underlying nerve meant to be undamaged.

FIGS. 4A-F illustrate five steps in decompressing a nerve by severing connective tissue, above the nerve, which is pressing on the nerve.

### DETAILED DESCRIPTION OF PREFERRED EMBODIMENTS

### The Instrument

The relative lengths of the handle region and the jaw regions can be varied depending on the amount of leverage the surgeon need to generate. The optimal ratio of the lengths of the handle region versus the jaw region will vary depending on the material used and the toughness of the tissue to be cut.

Similarly the overall length of both the instrument, as well as each component of the instrument will depend on the particular body location and the particular tissue(s) involved in the nerve decompression operation.

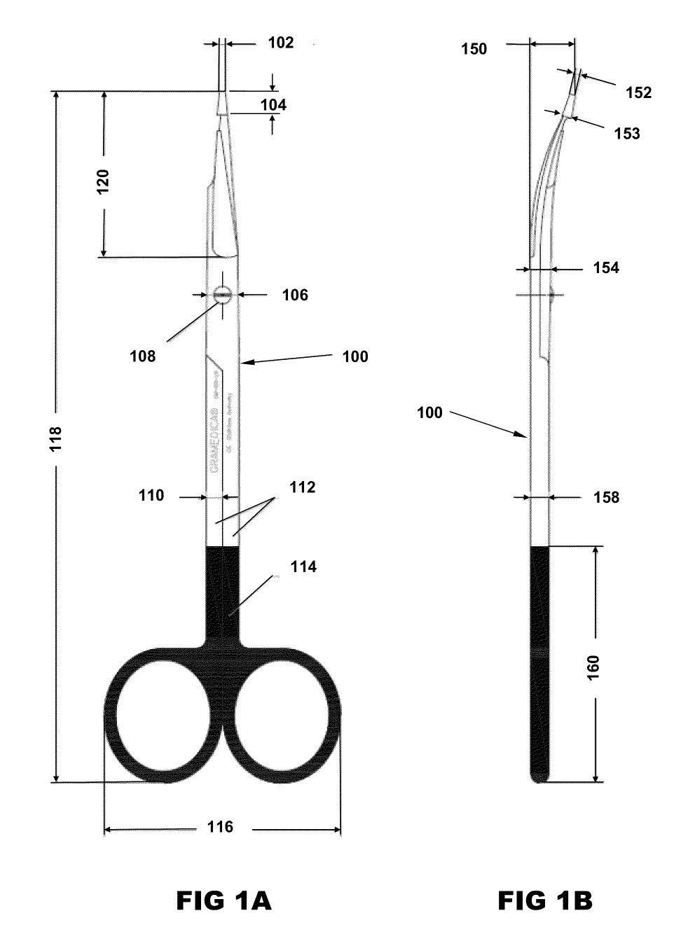

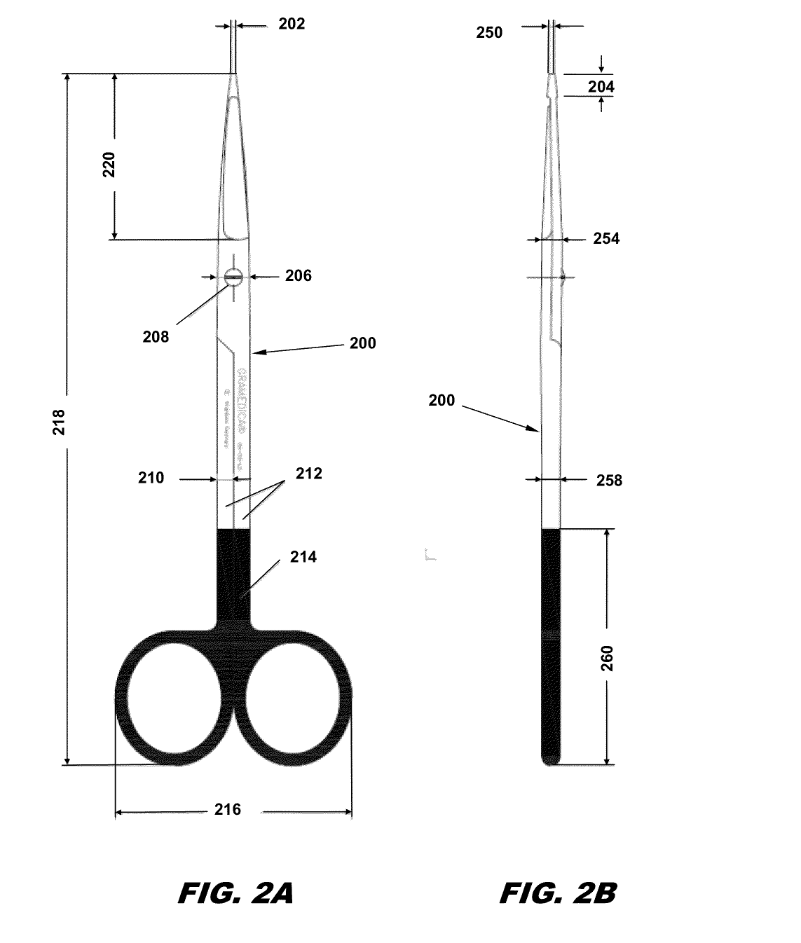

Various dimensions of the scissors are illustrated in FIGS. 1 and 2. These are a preferred embodiment of the scissors. Also contemplated are versions of the scissors in which these dimensions are 1-2 percent larger, 3-4 percent larger, 5-6 percent larger, 7-8 percent larger, 9-10 percent larger, 11-12 percent larger, 13-14 percent larger, 15-16 percent larger, 17-18 percent larger, 19-20 percent larger, 21-22 percent larger, 23-24 percent larger, and 25-30 percent larger.

Also contemplated are versions of the scissors in which these dimensions are 1-2 percent smaller, 3-4 percent smaller, 5-6 percent smaller, 7-8 percent smaller, 9-10 percent smaller, 11-12 percent smaller, 13-14 percent smaller, 15-16 percent smaller, 17-18 percent smaller, 19-20 percent smaller, 21-22 percent smaller, 23-24 percent smaller, and 25-30 percent smaller.

The inventor has found the following dimensions to be preferable. These dimensions provide ample leverage to sever the tissues compressing the nerve. At the same, the scissors are not so bulky as to interfere with proper placement nor will they visually impair the surgeon as he works.

Note the “thickness” of the scissors, at a given point, is defined as the measurement of the scissors taken in the plane perpendicular to the plane of the handles. The thickness is best seen in the view of the scissors shown in FIG. 1B. The “width” the scissors, at a given point, is defined as the measurement of the scissors taken in the plane parallel to the plane of the handles. The width is best seen in the view of the scissors shown in FIG. 1A.

Turning to the specific structures in FIGS. 1A-B, the overall preferred length is 146 with a standard error of 5 mm, or 146±5 mm. Note, unless indicated otherwise, the preferred standard error of a dimensions is 3 percent of the dimension. The preferred length from the tip to the pivot point is 42.5 mm. The length of the bladed region is 35±1.0 mm.

The tip of the scissors is made up of two jaws, a long jaw and a short jaw. The two jaws are designed to remove an upper lawyer of connective tissue but leave a lower lawyer of tissue undisturbed. The jays operate as follows. When the scissors are in the open position, the longer jaw is designed to be the lower jaw. The longer jaw contains a blunt, probe-like shape at the tip which is designed to enter the operation site first and, once there, promote separation of tissue without damaging that tissue. In a preferred embodiment, the operator inserts the tip between an upper layer of connective tissue and a lower layer of nerve tissue. The tip will promote a lifting and separation of the connective tissues from nerve tissue. After some degree of separation, the scissors will be further inserted into this space until the shorter jaws arrive in place above the connective tissue. Now, the connective tissue will be between the two jaws of the scissors. The scissors are then closed, severing the connective tissue but leaving the nerve tissue below undisturbed.

The probe-like tip of the long jaw is shaped like a blunt, narrow arrow head. The preferred length 104, from front end to back end is 5 mm. At the front end 102, 152 the arrow head has a preferred thickness 152 of about 1.2±0.1 mm. The arrow head widens to a 2 mm thickness at its back end 153.

This is in sharp contrast with the shape of the corresponding structure in prior art scissors (see e.g. D296,361; D372,783). These scissors are roughly the same size but the prior art tip is designed for a different function and has a different shape. These scissors are designed for cutting bandages. The tip is designed so that it can be pressed against the skin without puncturing the skin. This allows the tip to slide under a bandage which is wrapped around the skin. The upper jaw will move above the bandage. Then the scissors can be closed and the bandage is cut off. Accordingly, the tips of these prior art scissors are much broader than the tip disclosed herein. The prior art tips are all about 5 mm, several times broader that the tip disclosed here. Indeed, the prior art suggests the tip disclosed here would function rather poorly. The tip of this disclosure, if used to remove a bandage, would not only scratch the skin surface, but is so thin that it would trap hairs from the skin as the user attempts to cut the bandage away from the skin.

Another preferred embodiment is distinct from the prior art scissors, the novel curvature, shown in FIG. 1B. FIG. 1B illustrates a scissors displaced to the right 150. Note that the plane of the curve is distinct from that of the prior art scissors. Where the prior art scissors are curved in the plane of the motion of the jaws, they curve “up.” This is to that the scissors contact the skin at a highly oblique angle and this reduces the chance that the scissors will nick the skin.

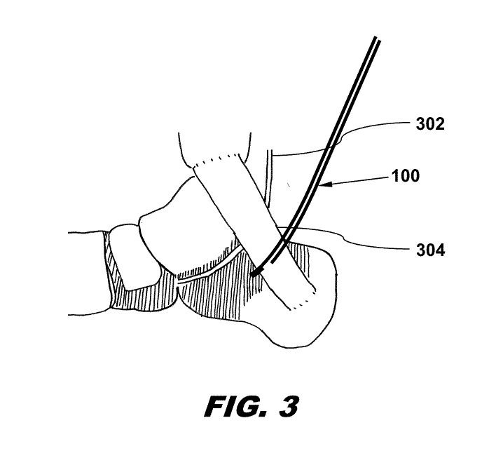

In sharp contrast, the disclosed scissors are curved in a plane perpendicular to that of the prior art, that is, they curve either to the left or to the right. The scissors are preferably curved, right or left, so that the tip is displaced 9.5±1 mm from center. The purpose of the curvature to promote separation of tissue without damaging underlying nerve tissue. This is illustrated in FIG. 3, a preferred embodiment showing the scissors at the medial (inner) side of a right ankle. The curvature of the scissors 100 follows the curved path of an underlying nerve 302. Indeed, the scissors and the nerve are substantially parallel. This design reduces the probability that the scissors will nick or otherwise damage the nerve by insuring that the scissors will only contact the nerve, if at all, at a safe, highly oblique, angle.

The prior art suggests a curvature to the right or the left would be utterly useless, even counter-productive. Unlike the prior art upward curvature, a right or left curvature would hinder rather than aid the prior art use of cutting and removing bandages from patients.

Turning now to the body of the disclosed scissors and FIGS. 1A-B and FIGS. 2A-B, the thickness preferably increases from 1.2±0.1 mm at the tip 102, 152 (see above) to 4.3±0.5 mm at the point where the two arms are joined 154. The scissor thickness then tapers off to a thickness of about 3.8 mm at a point halfway between the pivot point and the handles 158.

At the handles 116 the preferred width of the scissors is 50 mm. In the region 110 between the handles 114 and the pivot point 108 the width of each arm 112 is 3.5 mm for a total width of 7.0 mm. The width tapers down slightly from 7.0 mm to a preferred width 106 of 6.6±1.0 mm at the pivot point 108. The scissors remain at this width until the beginning of the bladed region where the width tapers down from 6.6 mm to a preferred width of 1.3±0.1 mm. The preferred length of the bladed region 120, 220 is 35±1.0 mm.

Also contemplated are scissors that curve in the opposite direction as those illustrated in FIG. 1. The curved scissors are designed specifically for one side of a patient's body. Accordingly, the pair of scissors, curving in opposite directions, are designed to treat both pairs of feet, left and right. It is contemplated that scissors curving in opposite directions will be provided together in a kit. It is also contemplated that the kit include the pair of curved scissors and the straight, un-curved, scissors.

The tip of the jaw having the blunt end extends approximately 7 mm beyond the tip of other jaw. The length of the blunt end is approximately 5 mm. The width of the blunt tip is approximately 1.2±0.1 mm.

### The Method

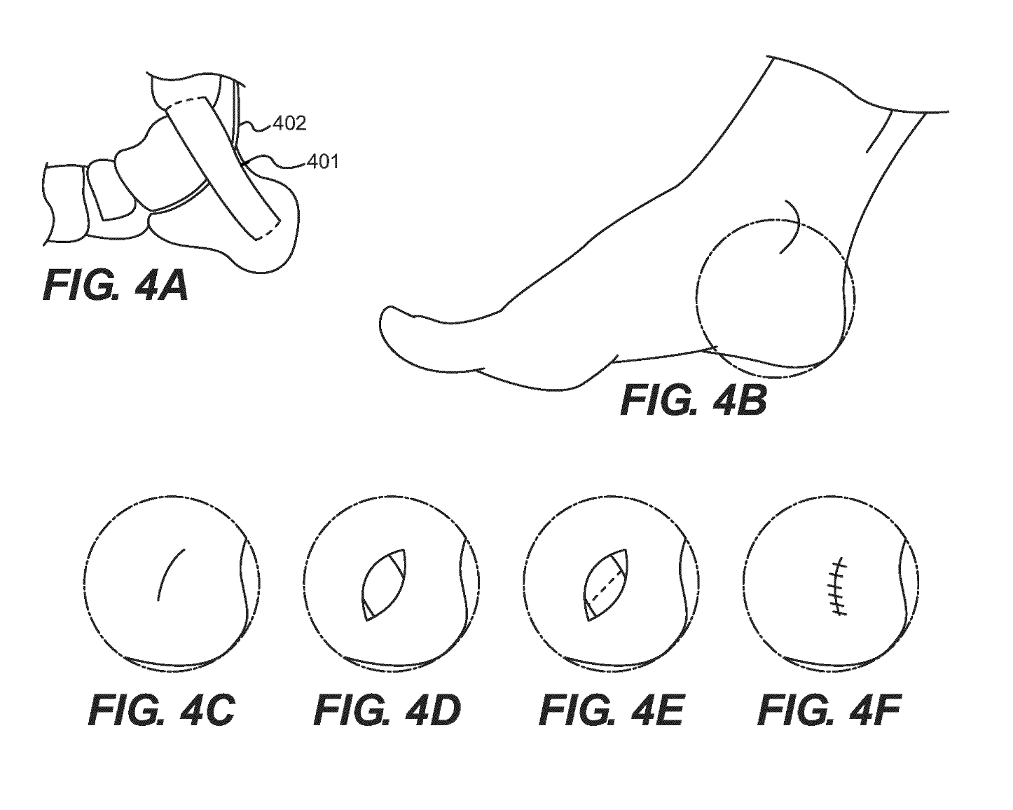

FIGS. 4A-F illustrate, in schematic form, a preferred method of nerve decompression. In FIG. 4A, the ankle structures are simplified so as to highlight the lacinate ligament 401 and the posterior tibial nerve 402. This particular nerve is located in the medial (inner) ankle region as shown in FIG. 4B. The method comprises: (1) making a small incision in the inner ankle region as shown in FIG. 4C, (2) exposing the lacinate ligament as shown in FIG. 4D, (3) cutting the ligament along the dotted line as shown in FIG. 4E. This is accomplished by positioning the scissors, illustrated in FIGS. 1 and 2, so that the lacinate ligament is between the jaws of the scissors. As the scissors are slid into place, the customized shape of the lower jaw keeps the posterior tibial nerve from becoming trapped between the jaws of the scissors. This is very important because the surgeon will be unable to see this nerve. The lower jaw tip will separate the nerve from the ligament above and the nerve will pass safely below the lower jaw of the scissors. After the ligament is cut, the ends of the ligament will retract thus releasing the pressure on the underlying nerve. Finally, the incision is closed as shown in FIG. 4F. As stated earlier, the smaller the incision the better. For several reasons this incision will be smaller when employing the above method. This is because only a single surgical instrument is used during the cutting of the lacinate ligament. Moreover, because the surgeon can be confident that the posterior tibial nerve will not be cut, there is no need to widen the incision in order to view the location of the nerve.

As described above the curved shape of the scissors is useful to avoid nicking nerve tissue. The methods described above are only one suggestion. The surgeon may chose to vary the angles slightly. The surgeon may also choose to insert the scissors from the opposite direction. In this case the surgeon may choose a scissors that curves in the opposite direction. Of course whatever the direction of curvature the surgeon uses on one foot, the surgeon will likely prefer the opposite direction when operating on the other foot. A surgeon may chose to avoid a curved scissors altogether. To promote maximum choice and flexibility, a preferred method of providing the scissors is to package the scissors into a kit of three: (1) one straight scissors, (2) one right curving scissors and (3) one left curving scissors.

In describing the invention, reference is made to preferred embodiments. Those skilled in the art and familiar with the disclosure of the subject invention, however, will recognize additions, deletions, substitutions, modifications and/or other changes which will fall within the purview of the invention as defined in the following claims.

I claim:

1. A surgical instrument for decompressing nerve fibers by excising tissue adjacent to said nerve fibers comprising:
two elongated arms,
said first arm comprising,
a first jaw region which further comprises a sharp bladed region for cutting tissue and longitudinally coextensive with the bladed region of a second jaw section, and, a distal region adjacent to said sharp bladed region comprising a blunt tip, and
a first handle region separated from said first jaw region by a pivot point, which further comprises a structure for grasping and manipulation by a surgeon
said second arm comprising,
a second jaw region which further comprises a sharp bladed region for cutting tissue, and
a second handle section, separated from said first jaw region by a second pivot point, which further comprises a structure for grasping and manipulation by a surgeon; and
said first arm and said second arm are connected at a pivot point,
wherein, said tip of said first jaw extends beyond said tip of said second jaw by approximately 7 mm and wherein said blunt end on said first jaw is approximately 5 mm long and approximately 1.2 mm wide.

2. A surgical instrument for decompressing nerves as defined in claim 1, wherein said first and second jaw sections have a convex curve in the plane of the scissors.

3. A surgical instrument for decompressing nerves as defined in claim 1, wherein said blunt tip region has a cross sectional shape selected from the group consisting of round, oval, round with flattened upper and lower portions and oval with flattened upper and lower portions.

4. A method of decompressing nerves comprising:
making an incision above the nerve to be decompressed,
exposing a tissue to be removed;
inserting a decompression scissors so that the tissue to be removed is between the jaws of the scissors but the nerve is not between the jaws,
cut the tissue to be removed, and
close the incision,
wherein said decompression scissors comprises:
two elongated arms,
said first arm comprising,
a first jaw region which further comprises a sharp bladed region for cutting tissue and longitudinally coextensive with the bladed region of a second jaw section, and, a distal region adjacent to said sharp bladed region comprising a blunt tip, and
a first handle region separated from said first jaw region by a pivot point, which further comprises a structure for grasping and manipulation by a surgeon
said second arm comprising,
a second jaw region which further comprises a sharp bladed region for cutting tissue, and
a second handle section, separated from said first jaw region by a second pivot point, which further comprises a structure for grasping and manipulation by a surgeon; and
said first arm and said second arm are connected at a pivot point,
wherein, said blunt tip is configured for and operable to separate tissue adjacent to nerve fibers from the nerve fibers and said blade sections are configured to cut and remove said adjacent tissue.

5. The method of claim 4 wherein said decompressed nerve is the posterior tibial nerve.

6. The method as defined in claim 5 wherein said incision has a length specifically designed to minimize the probability of wound dehiscence.

7. The method as defined in claim 6 wherein said incision length is large enough to allow cutting a lacinate ligament but not large enough to expose the tibial nerve.
